(19) United States
(12) Patent Application Publication
(10) Pub. No.: US 2009/0228073 A1
(43) Pub. Date: Sep. 10, 2009

(54) IMPLANTABLE MEDICAL DEVICE WITH PATIENT INPUT MECHANISM

(76) Inventor: Patrick Scholten, Lettele (NL)

Correspondence Address: MEDTRONIC, INC., 710 MEDTRONIC PARKWAY NE, MINNEAPOLIS, MN, 55432-9924, US

(21) Appl. No.: 12/044,264

(22) Filed: Mar. 7, 2008

(51) Int. Cl. A61N 1/08

(52) U.S. Cl. 607/60

(57) ABSTRACT

An implantable loop recorder (ILR) is subcutaneously implantable to record cardiac data. The ILR includes a patient activation mechanism whereby the patient can input commands to the ILR without using an external device. The patient activation mechanism may be a subcutaneous switch disposed on an outer portion of the ILR or a motion sensor that senses tapping of the device as an input. The patient input will direct the device to store data, telemeter data, or send a message summoning an emergency response. The ILR has distance telemetry capabilities so that a telemetered message is wirelessly transmitted to an external device which then relays the message to a remote location.

### FIELD OF THE INVENTION

The present invention relates to implantable medical devices. More specifically, the present invention relates to subcutaneous cardiac monitoring devices.

### BACKGROUND OF THE INVENTION

Various cardiac events and arrhythmias of the heart are difficult to diagnose based upon sporadic and infrequent monitoring, such as during in-office evaluation. These events, can be of short duration and sudden onset, coming with little or no warning, and may happen very infrequently. Holter monitors are well known for monitoring electrocardiograms for periods of time amounting to days or perhaps a week, but these are bulky and are applied externally to the body and interfere with the patient's normal life, making them impractical for long term use. Further, patient compliance cannot always be guaranteed, and is a common problem in use of the Holter devices Monitoring can be done using implantable pulse generators such as pacemakers and other heart stimulating devices or devices with leads in the heart for capturing physiologic parameters including the ECG. However, the expense and risk from implanting an intracardiac lead and/or a pacemaker with special monitoring functions is something both patients and physicians would prefer to avoid. Such devices, in addition to performing therapeutic operations, may monitor and transmit cardiac electrical signals (e.g., intracardiac electrograms) to an external diagnostic devices typically with leads fixed in the patient's heart, to observe electrical activity of a heart. It is common for implanted cardiac stimulation devices to send intracardiac electrogram signals to a monitoring device, such as an external programmer to allow a user to analyze the interaction between the heart and the implanted device. Often the user can designate that the communication from the implantable device to the programmer include a transmission of codes which signal the occurrence of a cardiac event such as the delivery of a stimulation pulse or a spontaneous cardiac depolarization.

In addition, there are subcutaneously implantable monitoring devices that collect and record data over a longer period of time, then telemeter some or all of this data to an external device during an interrogation. An example of such a device is the Medtronic Reveal™ implantable loop recorder. The following references related to subcutaneous monitors and are herein incorporated by reference in their entireties: U.S. Pat. No. 5,205,283 to Olson, issued Apr. 27, 1993, entitled “Method and apparatus for tachyarrhythmia detection and treatment,” U.S. Pat. No. 5,233,984 to Thompson, issued Aug. 10, 1993, entitled “Implantable multi-axis position and activity sensor,” U.S. Pat. No. 5,312,446 to Holschbach et al., issued May 17, 1994, entitled “Compressed storage of data in cardiac pacemakers,” U.S. Pat. No. 5,331,966 to Bennett et al., issued Jul. 26, 1994, entitled “Subcutaneous multi-electrode sensing system, method and pacer,” U.S. Pat. No. 5,987,352 to Klein et al., issued Nov. 16, 1999, entitled “Minimally invasive implantable device for monitoring physiologic events,” U.S. Pat. No. 6,230,059 to Duffin, issued May 8, 2001, entitled “Implantable monitor,” U.S. Pat. No. 6,236,882 to Lee et al., issued May 22, 2001, entitled “Noise rejection for monitoring ECG's,” U.S. Pat. No. 6,412,490 to Lee, issued Jul. 2, 2002, entitled “Tool for insertion of implanatable monitoring device and method,” and U.S. Pat. No. 6,317,626 to Warman, issued Nov. 13, 2001, entitled “Method and apparatus for monitoring heart rate.”

### BRIEF DESCRIPTION OF THE DRAWINGS

FIGS. 1 and 2 are the exterior side view, interior block diagram, respectively of a prior art device.

FIG. 3 is a block diagram illustrating the main circuit and assembly of a device in accord with a preferred embodiment.

FIGS. 3A-D are block diagrams of preferred embodiment circuits of the implanted device used for monitoring and storing ECGs.

FIGS. 4a, 4b, and 4c are exposed front, side, and back views, respectively of a preferred embodiment of the invention.

FIG. 5 is an illustration of a preferred embodiment of the invention, showing (in dotted line), locations for fin/wing and stubby lead features.

FIGS. 6a and 6b are front and side views of preferred embodiment cross-sections taken from FIG. 5.

FIGS. 7A, and 7B are front, and cross section views of another preferred embodiment of the invention.

FIG. 8 is a front view of another embodiment of the invention.

FIG. 9 is a block diagram of the looping memory and its control circuitry in accord with a preferred embodiment of the invention.

FIG. 10 is a flow chart of the functioning of the recordation of triggered events in a preferred embodiment of the invention.

FIG. 11 is an illustration of an implantable medical device having a subcutaneously accessible switch.

FIG. 12 is an illustration of an implantable medical device having a sensor for sensing patient tapping as a communicative input.

### DETAILED DESCRIPTION OF THE PREFERRED EMBODIMENT

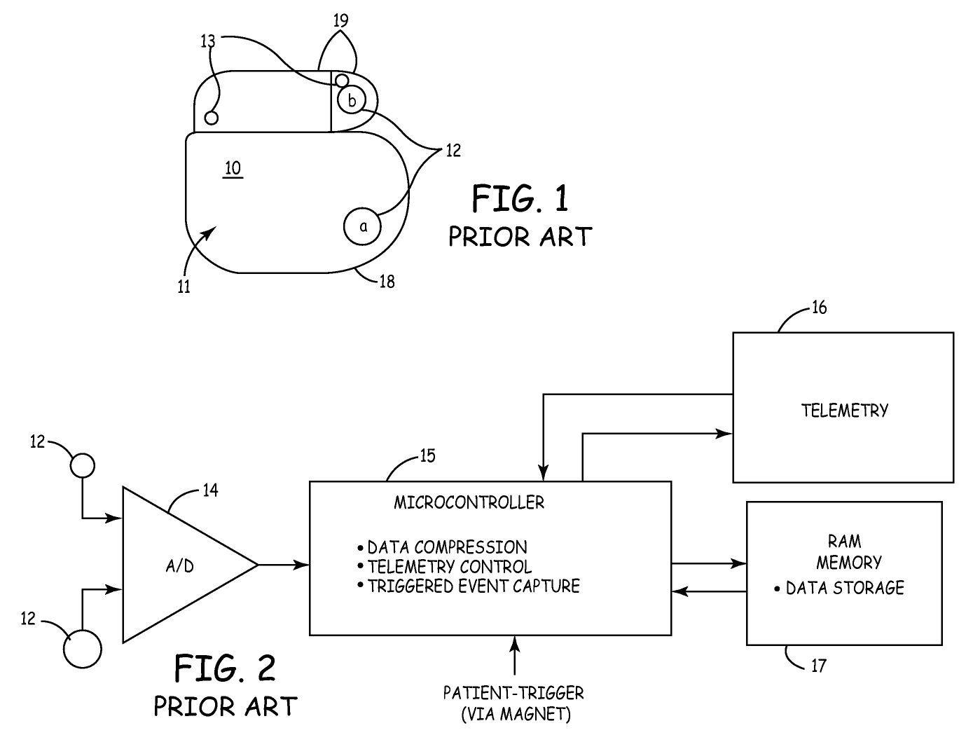

With reference to FIG. 1 a device 10 is provided with two suture holes 13 and two spaced apart non-lead or leadless electrodes 12 at one and one-quarter inches distance center to center. The device 10 includes a coating 11 so that the only area of exposure on the body of a pacer can 19 is the exposed area at the electrode 12a. The other electrode is a metal plug electrode 12b mounted in a connector block 19.

In FIG. 2 the same electrodes 12 supplied signals into the circuitry inside the housing or “can” 18 (FIG. 1) by first entering a analog to digital conversion and amplifier circuit 14. Data from this circuit 14 was fed to a microcontroller 15 which provided functions of data compression, telemetry control and event capture triggered by patient operation. Telemetry block 16 and RAM memory storage 17 were also provided in this device.

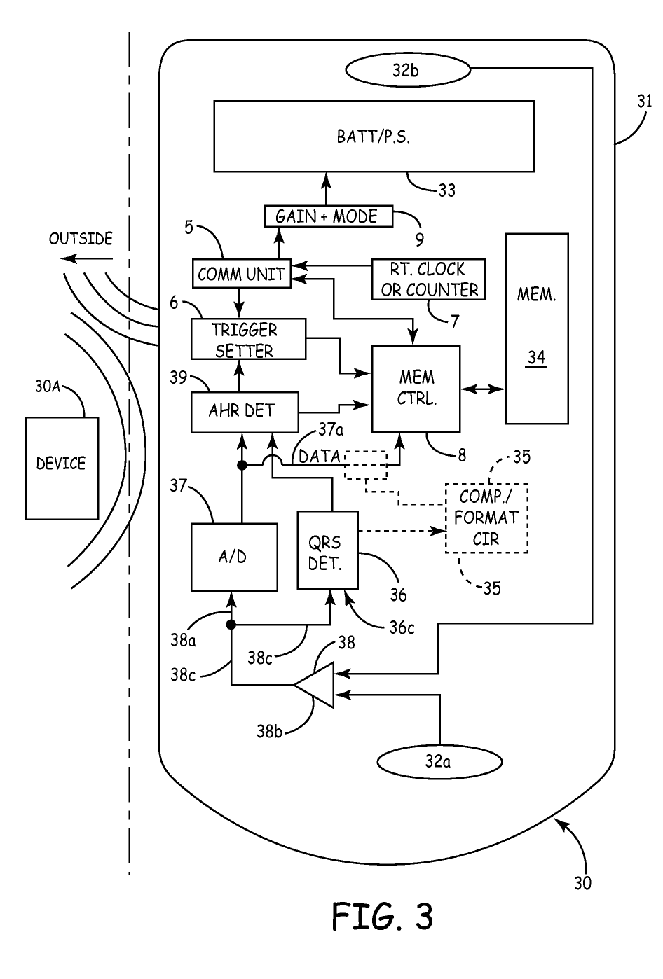

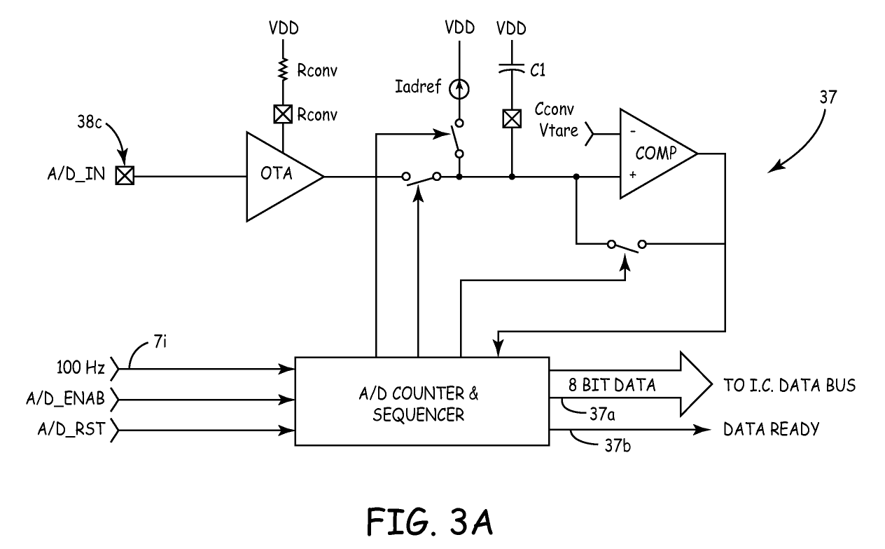

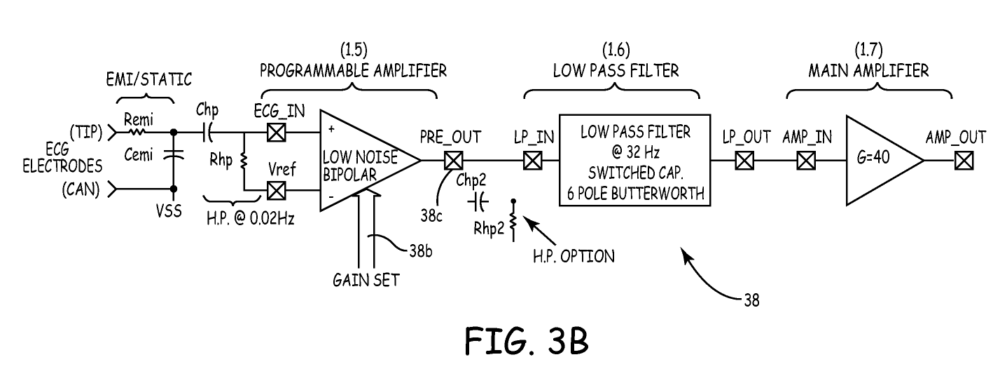

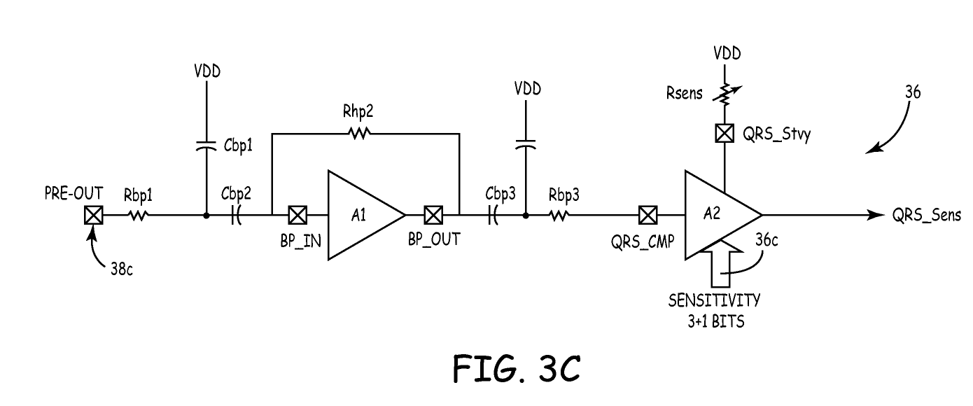

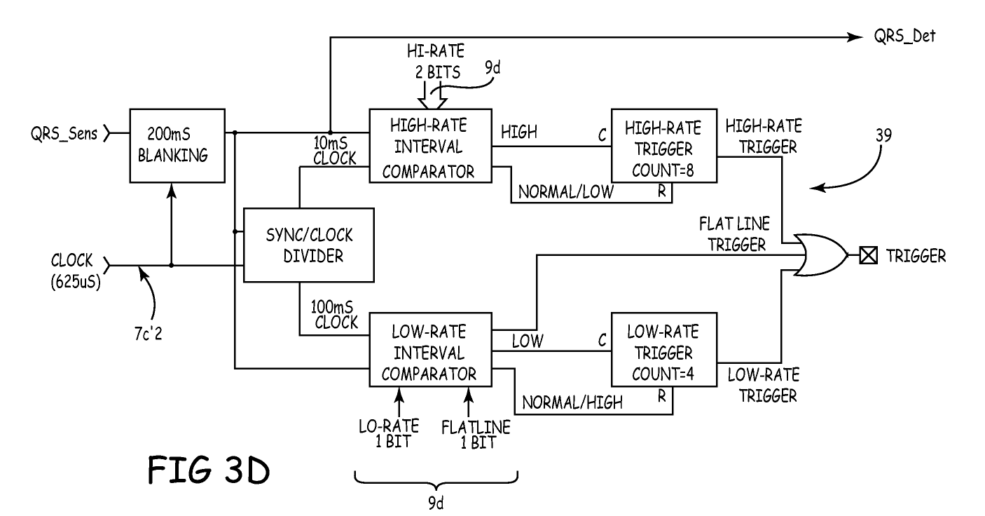

Referring to FIG. 3, a circuit model 30 is illustrated in an outline of an implantable device shell 31. Electrodes 32a and 32b bring signal from the body to an input mechanism 38, here drawn as a differential amplifier for simplicity only, the output of which is fed to a QRS detector 36 and an A/D converter 37. Both these circuits 36 and 37 supply output to an arrhythmia detector 39, which in this preferred embodiment supplies the autotrigger signal to the trigger setting circuit 6. The data output from the analog to Digital converter may be converted, compressed, formatted and marked or reformulated if desired in a circuit 35 before the data is ready for input into the memory 34. The memory control circuits 8 receives input from the A/D converter, with or without conversion and so forth from circuit 35, from the auto triggering determination circuit (here seen as the arrhythmia detection circuit) 39 (which may include input directly from the QRS detector if desired) as well as signals from the trigger setter circuit 6. The trigger setter circuit may also be controlled by a communications unit 5 which operates to receive and decode signals from the outside of the implant 30 that are telemetered or otherwise communicated in by a user. This communications unit 5 will also be able to communicate with the memory controller to request the offloading of memory data for analysis by an outside device. It should contain an antenna a or other transceiver device or circuitry to communicate with an outside device such as device 30A. A clock or counter circuit 7 reports the time since start or real time to the outside interrogator device 30A contemporaneously with a data offloading session so that the events recorded in memory 34 may be temporally pinpointed.

Alternatives to this overall design may be considered, for example by using a microprocessor to accomplish some or all of the functions of circuits 6, 8, 39, and 35

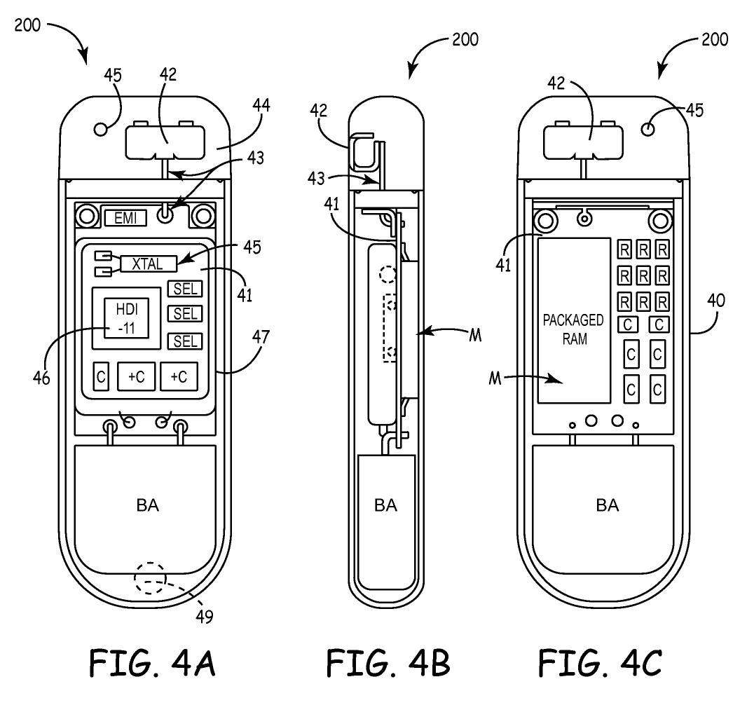

FIGS. 4a-c illustrate one configuration of the invention embodied as implantable medical device (IMD) 200. In this form IMD 200 has an outer titanium shell 40, in a plastic cap means 44, which together form the exterior of the device. The cap means 44 may be composed of material similar to those used for pacemaker connector blocks as it is in the case. The two electrodes, 44 and 49, provide metal surface contacts to the body. Electrode 49 is formed as a whole in a parylene coating over the metal body 40, of the device. The metal electrode 42 is connected via a feedthrough 43 which is itself electrically connected to the circuit board 41. Circuit board 41 contains all the electronics required for the device function and is connected to a battery BA for power. An integrated circuit 46 houses circuitry and intelligence required for the function and the memory M is packaged on the other side of the circuit board. In this preferred form, the invention uses a communications circuit 45 having a telemetry antenna both to indicate from outside the body that a read out is requested of the device, and for communicating data out from said device. Programming of the device or mode setting will also use the communications circuit 45. The communications circuit 45, in some embodiments, includes an RF transceiver capable of communicating with an external medical device 30A over ranges from a few to e.g., 20-30 meters. This so-called distance telemetry provides for wireless communication between the IMD 200 and the external medical device 30A to permit programming or the uplinked transmission of data collected from the IMD 200. The external medical device 30A may be a medical device programmer as previously described, a home monitor which is a stand-alone device that provide a communication link between the IMD 200 and a remote device such a central server or a personnel communication device. The personnel communication device also provides a communication link between the IMD 200 and a remote device. For example, the personal communication device could be an analog or digital cellular telephone, a PDA, a pager or any electronic device configured to receive communications from the IMD 200 and access a communication pathway to another device and/or a caregiver.

In this form also a suture hole 45 is provided through the cap means 44. Electrode 49 is connected by a conductive connection (not shown in this fig.) to the circuit board. In this embodiment the length “I” is 2⅜″ and “w” is ¾″. These measurements can be varied within the constraints described. Electrode spacing here is about 1¾″, center to center.

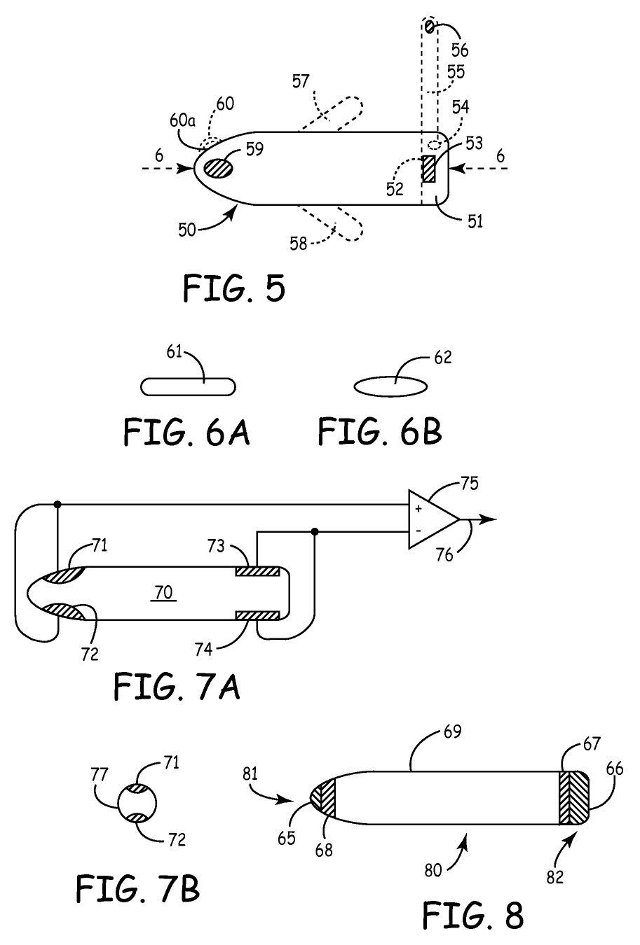

Three or more electrode embodiments are also described with reference to FIGS. 5-8. A third electrode, like electrode 56, can be used to optimize signal strength responsive to changes in device position, heart position, or body position. A transistor or other switch means can switch the electrode configuration automatically based on a determination of signal strength or direction from an outside device through the communications circuit. In order to retain the elongated shape yet provide a well spaced orthogonal position, the third electrode can be mounted on a self-positioning (flexible, rigid, or semi-rigid) stubby lead. An additional variation from the most preferred design could provide for a wing or fin-shaped member 57 or more than one wing (57, 56) that extend substantially in one plane from the main body of the device. Ideally this would be approximately in the same plane as the other two electrodes (53 and 59). Unless they are constructed so as to spring from the main body outward after insertion into the intended body area, wings like 57 or 58 will require a larger incision than required for a smooth bodied device. The illustration of the device 50 in FIG. 5 without the dotted line external parts 55, 57, 58, and 60.

A single suture hole 54 (or two or more if desired) can be provided in the cap. Additional suture appendages, like ring 60, having a suture hole 60a, may additionally be provided for more stability. Additionally, a suture may secure the stubby lead (if present) to the patient's tissue if desired. These suture holding means allow the device to be fixedly held in one orientation in the body of the user, whether intramuscular or strictly subcutaneous. Intramuscular pocket implantation is advantageous in that the device may be protected form the outside world by a layer of muscle, which will provide cosmetic benefits to the patient as well. The exact sites of implant may advantageously be varied from patient to patient for various reasons apparent to the physician. Implant just under the skin now appears to provide the signal most free of skeletal muscle myopotential or body movement signal interference.

While considering the features of the embodiments illustrated by FIG. 5, it is well to note the electrode configuration. Here the electrode 53 is a conductive or metal plate compatible with the patient's body that is on one surface of the cap unit 51, the cap being delineated by dotted line 52. One can construct the device 50 as a solid container having out of body compatible materials. For examples, titanium or other metal or alloy, coated with compatible insulator but exposed for at electrode areas or fitted with conductive electrodes. This distance should be at least far enough to achieve good signal measurement but not too far so as to make the size of the implant unnecessarily large.

In the present embodiment the cross-section of the device is an easy-to-insert rounded rectangular or oval shape that also reduce the ability of the device to turn over after implant. FIG. 6A shape 61 and FIG. 6B, shape 62 illustrate this concept while any similarly functional cross-sections may be substituted.

Additional features are illustrated which can assist in preventing medically unintended movement of the device. In FIG. 7A the electrodes are placed so as to be matched on opposite sides of the rectangular, round, or ovoid shaped device and electrically connected in parallel on opposite sides to retain the same signal in spite of flipping or other movement. (The internal circuitry would operate like the op-amp 75 to produce output 76 from electrodes 71-74 as shown to produce this effect.) In surface pacemaker implants, patient populations have been known to play with their implants, often unconsciously and this has been a common enough problem in the pacemaker art to have obtained the name twiddler's syndrome.” These features address this problem. The device of 7A is seen in cross-section in FIG. 7B.

Another embodiment employs circumferential electrodes on a cylindrically shaped device. In FIG. 8 this device can be seen to also have a body 69 that is tapered on one end 81 and blunt on the other 82. The effect again is to provide a constant signal in spite of likely unwanted rotation of the device, because the electrodes each extend around the device circumference. Here the electrode area positions are illustrated for each end, 65 and 68 for end 81 and positions 66, 67 for end 82. This approach trades-off the protection from muscle noise of the rectangular outward-facing device.

In FIG. 3 the inventive system is described as stated above. The external device 30A is preferably a device that is commonly called a programmer in the pacemaker art, because it's usual function is to communicate with and program implanted devices. Software modifications and modifications to the telemetry system of device 30A to accommodate communication with and analysis of data from device 30 can be made as required. Such modifications will vary with the programmer type and are within the discretion of the manufacturer and thus will not be illustrated here. Using a programmer will avoid having to have additional devices cluttering the operating room or clinic by creating a separate and distinct external communications device for this invention. The functionality necessary for mere ECG monitoring and event triggering is minimal, so in the preferred embodiments that only monitor some form of ECG or other limited sensory input, a microprocessor can be and is done away with altogether by using particularized functional circuits instead of doing the functions in software.

In FIG. 3A, a block diagram of an analog to digital conversion circuit for use in this invention is shown. The clock input may advantageously use an output from the clock circuit 7, input 7i. The input 38c is the analog input signal from input circuit 38, and the converted output is a stream of 8 bit digital data words on line 37a, sequenced by a timing line 37b.

FIG. 3B illustrates the basic parts of circuit 38, additionally indicating the input of gain set bits which can modify the value of the output of the low noise bipolar amplifier for output at line 38c, the input to the QRS detector. In this invention QRS detection is done on the analog signal, advantageously saving more complex detection after digital conversion.

In FIG. 3C QRS detect circuit 36 has a 2nd order bandpass filter with a center frequency preferably in the 20-25 Hz range. It includes a transconductance amp A1, summing amp/comparitor A2 and resistors Rbp1-3, capacitors Cbp1-4 and selectable resistor R sense connected as shown. R sense is preferably adjusted during manufacture. Additional control is provided for QRS sensitivity at line 36c, since the gain is delectable for this input.

A simple arrhythmia detection circuit 39 is included with this preferred embodiment, and illustrated in FIG. 3D. The output from circuit 36 is monitored the preferred embodiment, a high rate can be selected amongst 4, with two selection bits dedicated to do so at input 9d and the low and flatline trigger rates each have one bit to turn them on or off provided by inputs 9d. These inputs designated 9d preferably come from a register that holds the gain the mode and the rate settings, illustrated as register 9 in FIG. 3. Such features may be programmable through communication with the implanted device by an external device. Preferred timing for the high rate triggers is 140, 162 and 182 beats per minute, requiring 8 consecutive beats at such a rate to initiate the trigger. Additionally the trigger may be programmed off. The low rate counter/comparitor may be programmable to detect low rates of 40 or 30 bpm, requiring 4 consecutive low rate intervals to trigger. Additionally a flat-line trigger can be set to occur after 3 or 4 and one half seconds of no QRS detection.

For embodiments that include more sensors and/or electronics, an additional sensor could be added to benefit the patient. One particularly useful would be an activity sensor based on a single or multi-axis accelerometer, which indicates the level of patient activity and his orientation. By checking for output that indicates the occurrence of a VVS (VasoVagal Syncope) episode, (for example, the patient falling from an episode) such an addition offers an improved trigger for events that might otherwise be missed by an arrhythmia detector set up like in FIG. 3D. Such a sensor trigger could replace the circuitry of 3D.

Additional circuits may be provided to support additional functions if desired, however in order to reduce size and power consumption and extend the life of the device and reduce the intrusion into the body of the wearer, auxiliary circuits should be kept to a minimum. Such additional circuits could support temperature sensing, oxygen sensing, pressure sensing, respiration sensing, and any other kind of sensing that can be demonstrated to have been known for implanted devices. They may each have their own auto triggers based on sensor output, or depend on manual triggers. Additionally, activity sensing or positional sensing devices can provide additional input for recordation and or autotriggerring functions. As new sensors become available they may also be incorporated into these designs.

One function of the various embodiments of the present invention is the long term ECG monitoring of the subcutaneous (or intramuscular) ECG. The device continuously records and monitors the subcutaneous ECG in an endless loop of memory. In its primary mode the device is triggered to save/retain in memory the last X minutes or seconds of ECG data by the patient subsequent to feeling symptoms of interest (e.g. syncope, palpitations, etc.).

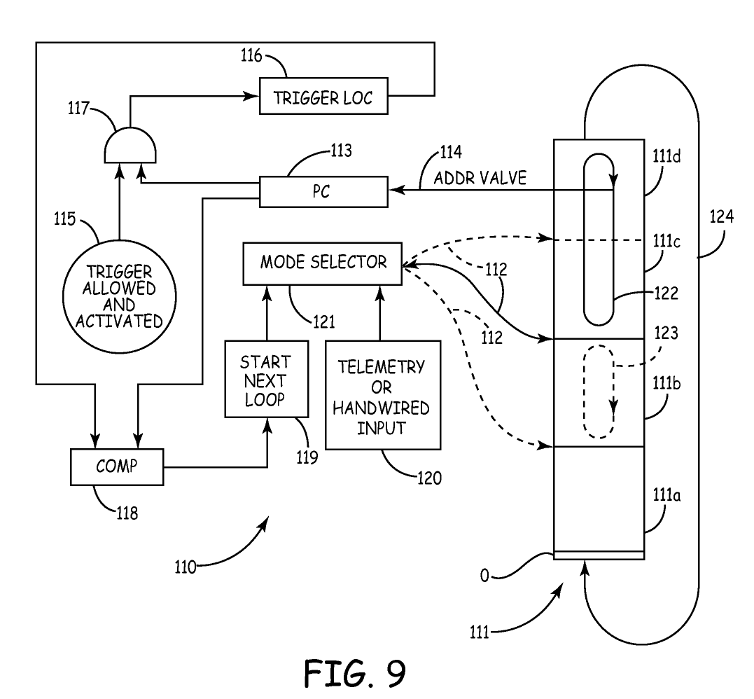

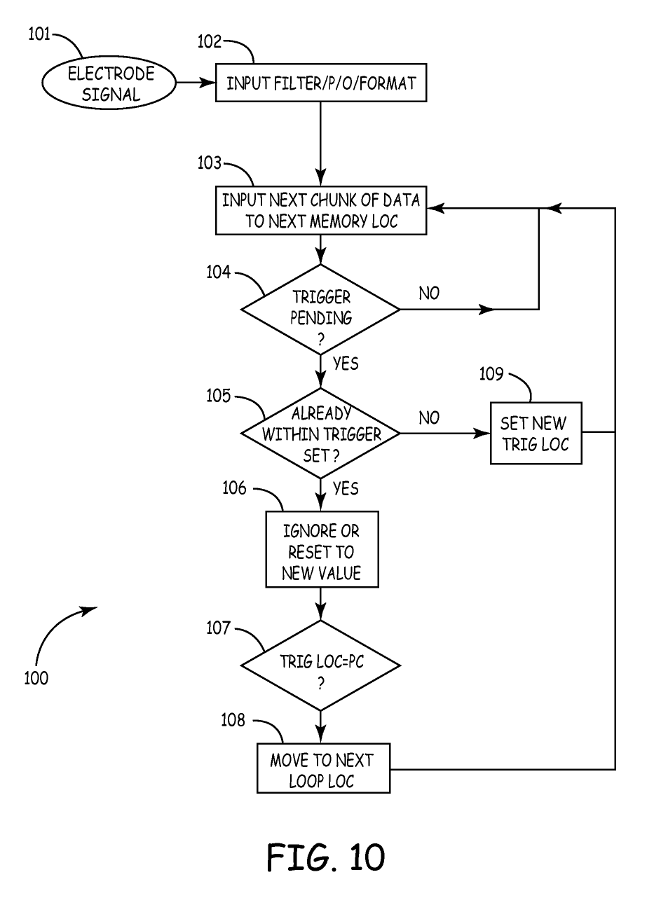

Additional modes include those with pure autotriggering, which can mirror the patient triggered modes if desired. It should be considered that with autotriggered events, the determination by the device of an event worth recording and the subsequent activation of the trigger by the device itself will be faster than the patient finding his device for activation or otherwise activating the device, so the pre trigger time record can be smaller. In one preferred embodiment the memory is segmented to allow for 14 autotriggers and 3 manual triggers. Further detail regarding modes is described with reference to FIGS. 9 and 10.

The patient activated triggering of a preserved form of the recorded ECG signal can be carried out by using a small handheld external device which may be of any number of different forms. A first way is through a handheld battery-powered device which uses a coded radio-frequency telemetered signal through the skin to the device, on the press of a button. Alternatively, a small handheld device having a magnet is used to close a magnetic switch within the implanted device to trigger it by holding the magnet close or patting the area of the body that has the implant a set number of times with the magnet. Other methods for triggering ECG data retention in memory (each of which has it's own advantages for implementation) are to use physical tapping or slapping of the finger or hand on the skin over the device in a particular cadence and/or number of taps. With such methods the disadvantage is that the patient needs to memorize the triggering sequence. Matched voice activation with a known command is possible but the complexity at this time of discerning voice commands precludes such activation for the present time, but could be in future devices using this invention. Another approach is light activation through the skin using a light source and receiver, auditor/sonic activation using a handheld auditory sonic source held over the skin with a microphone receiver in the device. All these methods are patient activated and require patient compliance or cooperation, a feature this device was designed to avoid. Accordingly, in conjunction with one of these patient triggers or alone, an automatic activation or trigger for holding a chunk of memory should be included. This could be activated by automatic recognition of an arrhythmia, a heartbeat too fast or too slow, or for any other condition the device may be set up to find.

If a patient trigger is used it is advantageous provide feedback to the patient regarding whether the attempt to trigger long term storage of the event was successful. To accomplish this, the implant should telemeter out a signal that indicates it has recognized a valid trigger. (This of course requires additional circuitry and usage of the limited available power supply.) The external triggering device then notifies the patient via the triggering device or through some known alarm mechanism whether they have or have not properly triggered the implanted device. This notification can be one of any combination of a number of feedback methods including: one or two visual sources such LED's, an auditory source such as a beeping speaker in one or two tones, or a tactile source such as a vibration.

Referring now to FIG. 9 in which a block diagram of a functional model 110 of the controller and memory 111 of an embodiment of a device is illustrated. The memory is generally organized as a continuous loop of, preferably, 8 bit addresses starting at address 0 and looping back around to address 0 through line 124. By telemetry or hard-wired input during manufacture 120, a mode selector 121 is set so as to divide the memory 111 into working segments 111a-d. The address of the start of each of these segments is indicated with lines 112.

Since this device is used for recording physiologic data, after the data is compressed, converted, formatted and is in appropriate digital form, it is continually recorded in the memory 111. The address value at the tip of arrow 122 in the combined memory space 111d, 111c is monitored by a program counter register 113.

The size of each memory segment set in a given mode limits the amount of data available for each triggered event. In the preferred embodiment, using only one program counter set of registers, the flexibility to accommodate two different trigger lengths can be limited. Alternate forms of memory allocation are available. For example organizing the entire looping memory as one unit and marking Mach trigger would allow more flexibility but increase the overhead. See for example the memory structure in Enigra, U.S. Pat. No. 5,339,824, FIG. 7, incorporated herein by reference in its entirety.

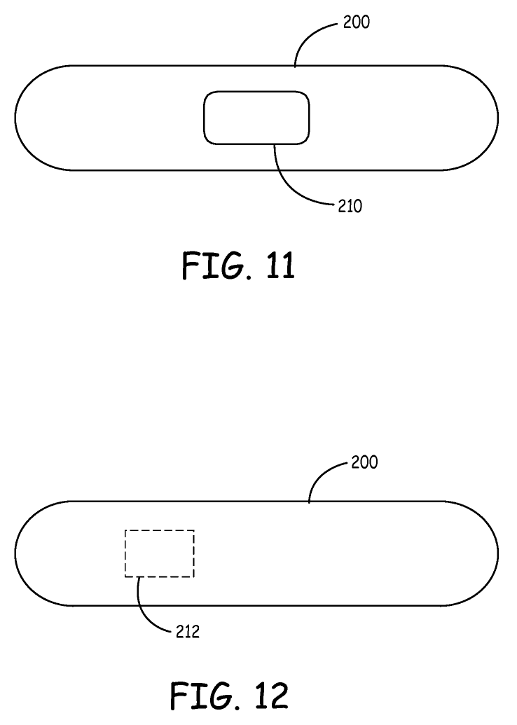

To use a single program counter the actual trigger address minus the time (in memory location storage events) required to have already stored the amount of data needed for prevent analysis for that trigger is stored as a value in the trigger location register 116 of FIG. 11. If a larger time for pre trigger recording is required by a trigger occurring during an already triggered event, (say, a manual trigger follows the occurrence of an auto trigger), the value in the trigger register can be decremented, thus yielding a larger pre trigger time period in the allocated memory segment for this event. A priority system for whether to extend the pre trigger record is simple to implement but again would require additional hardware and is not preferred. In fact the simplest construction ignores any new triggers once a trigger is set until the results of comparing the program counter with the trigger register corresponds to a match in value.

It is preferred to save more data for a manual triggered event than an auto triggered one because upon recovering from an event the patient has enough time to recover, get their wits about them, and find the triggering device. Manual triggering may therefore be set to record in double or multiple sized segments. FIG. 9's segments 111c and d are joined by looping arrow 122 to give effect to this concept.

Because the memory size is limited a time record or first-in-first-out protocol should be kept on order that the newest triggers record only over the oldest events segments. An additional preferred feature allows for a mode that prevents recording over any triggered event segment. This is preferably implemented by a counter which fills for each segment used and has storage for the set number of looping segments. When it is full recording of new events stops.

When a trigger is activated and under the control program of the device is allowed, a signal 115 is permitted by some control gate 117 to allow the program counter address to be loaded into a trigger location address register 116. After loading, each subsequent clock cycle or set of clock cycles depending on the configuration of the device will load the trigger location from 116 into a comparator 118 to compare this location with the program counter address stored in register 113. When comparator 118 finds that they match, an appropriate output is generated to start the next loop via control circuit 119. This control circuit 119 will cause the mode selector to point to the next available loop location effectively placing that into the program counter 113.

The diagrammatic algorithm 100 to indicate the flow of this information is found in the illustration of FIG. 12 in which an electrode signal 101 is input filtered, converted from analog input to digital values, compressed and formatted if desired in step 102 so as to be in appropriate form to store in a memory location designated by a program counter pointer.

This data word's form could be containing a value representing input signal compressed at various available ratios, and may be mixed with other information like data provided by another sensor or clock data. The data stored will of course carry information related to the signal taken at the sampling rate. Thus lower sampling rates to save power will adversely affect the usefulness or detail of the data. Whatever its preferred form each data point stored as a word is referred to as a chunk.

Output form step 102 provides the next chunk of data to the next memory location in step 103.

Device checks to see if there is any trigger pending after storing each chunk of data in step 104. If not, the next chunk of data is stored. If there is, the device preferably checks to see if there is another trigger already set and if so either ignores it or resets the value of the reserved looping memory area (like areas 111a-d in FIG. 9) to accommodate a larger trigger or it ignores the trigger if it is smaller or if it indicates a smaller value needs to be stored. If on the other hand, no trigger is already set, then a new trigger location is recorded in the trigger location memory and then the next memory location is written with the next chunk of data. At step 107 if the trigger location is equal in value to the program counter, the device knows that it has gone through the entire loop reserved by the mode selector for this particular event record and then moves on to the next loop location, step 108.

It should be recognized that any of the inventive concepts taught herein may be applied to implantable devices to supplement their other functions, such as a supplemental recording system for a pacemaker, implantable drug pump, et cetera. Further, known enhancements to telemetric communication can be used to automatically activate offloading of data to a device located in the patient's home. Such a device could send its received communications to the attending care giver/physician's office at some convenient time, telephonically or otherwise so as to enable close compliance with prescribed follow-up of patient conditions. This invention is not understood to be limited in scope except by the following claims.

FIG. 11 illustrates an embodiment of IMD 200 further including a subcutaneously accessible switch 210. FIG. 12 illustrates an embodiment of the IMD 200 further including a motion sensor, such as an accelerometer 212 disposed within the device. The switch 210 or the accelerometer are provided to permit patient actuation of the IMD 200 while the device is implanted without requiring the use of any external handheld device, such as a programming magnet or RF activator. Thus, patient activation may be achieved by tapping or pressing on the surface of the skin over the implant site. This will either depress the switch 210 or the motion will be detected by the accelerometer, depending upon the embodiment.

There may be multiple commands for the patient to select from. For example, one command would indicate that the patient is experiencing symptoms and that the IMD 200 is to save the recorded data for some predetermined period of time prior to actuation and following actuation. Another command may be an indication that the symptoms are severe. This would not only cause the IMD 200 to record data, but also to immediately begin to attempt communication with the remote device via the external medical device 30A. In one embodiment, this entails transmitting the collected data to a remote server for immediate review by a caregiver. Upon review, the caregiver may instruct the patient to take certain actions such as altering a medication regimen or indicating that follow up care should immediately be sought. Alternatively, or in combination, the caregiver identifies this as an emergency situation and summons a medical response to the patient's location.

Similarly, whether due to the typical symptoms sought to be recorded by the IMD 200 or due to some other situation, the patient may wish to summon emergency care to their location. Thus, the patient activation initiates a communication via communications circuit 45 to the external medical device 30A which in turn sends a communication to a remote device (e.g., a 911 phone call, an e911 message, or some other automated message) to indicate that the patient is in need of emergency assistance. Thus, the patient having the IMD 200 implanted may respond to an emergency situation and summon help simply by tapping on or otherwise actuating the IMD 200, without the need for an external patient actuator.

As described, the IMD 200 is only capable of transmitting to an external medical device 30A located within a range of, for example 3-30 meters. Thus, the external medical device 30A, whether in the form of a home monitor or a patient worn or carried device would need to be within range to complete the transmission. This is due to the limited transmission capability of the communication circuit 45. As technology improves, it may be possible to incorporate the capability of connecting with a longer distance transmission format within communication circuit 45 and thereby obviate the need for the external medical device 30A.

In practice, the use of the subcutaneous switch 210 may require the patient to actuate the switch once to indicate symptoms should be recorded; twice to request immediate data transmission; and three times to signal an emergency condition and request a response. Several mechanisms may be utilized to avoid or minimize inadvertent actuation of the switch. For example, the switch may be somewhat recessed so that firm pressure by one or two fingers is required. This would minimize inadvertent actuation due to laying down, crossing ones arms, or other normal physical contact. Alternatively, or in addition, a confirmation actuation may be required. For example, after depressing the switch the desired number of time, the patient may have to wait for a predetermined period of time or for an audible or vibratory response from the IMD 200 and then press the subcutaneous switch 210 yet again to confirm that the actuation was intentional.

Similarly, use of a sensor 212, such as an accelerometer, to detect deliberate tapping on the device could employ various patterns. For example, three quick taps would indicate that symptoms are presents; five taps could request immediate transmission; and seven could summon an emergency response. Again, there is a need to balance simplicity and ease of use against inadvertent actuation. Thus, preferably a pattern of taps is required for actuation. Alternatively or in addition, once the IMD 200 recognizes patient actuation via tapping, a secondary input may be requested or required as confirmation. For example, the patient may need to way a predetermined period of time and actuate the device again. Alternatively, the IMD 200 may generate a signal such as an audible tone, recorded message, or vibratory signal. In response the patient may actuate the device to confirm the validity of the input.

In either embodiment, the IMD 200 will monitor patient parameters, such as the collected ECG data. In the event an emergency response was presumptively requested but the patient failed to confirm the actuation, the IMD 200 may initiate the request for an emergency response based upon the monitored patient parameters. In this manner, the IMD 200 will not require patient confirmation to summon an emergency response when an appropriate medical condition is detected. Similarly, the IMD 200 may initiate the communication to summon an emergency response even in the absence of any patient request if an appropriate medical condition is detected, as the patient may not be able to actuate the device.

It should be appreciated that other forms of patient actuation are possible that do not require the use of a device separate from the IMD 200 and are included within the scope of the present disclosure. Further, the above described embodiment are only illustrative of type of patient controlled inputs and are not meant to be limiting in either the type of message input nor in the form, format or pattern of the actuation communication.

1. An implantable medical device (IMD) comprising:
a housing;
control circuitry disposed within;
at least one electrode disposed on an outer surface of the housing and coupled with the control circuitry for sensing a physiologic parameter;
a memory coupled with control circuitry for selectively recording data from the electrode;
an actuator contained within the housing and configured to provide a post-implant patient data input mechanism through a mechanical actuation to the control circuitry.

2. The IMD of claim 1, wherein a first mechanical actuation signal causes the actuator to generate a first actuation signal that causes the control circuitry to store data in the memory.

3. The IMD of claim 1, further comprising:
a telemetry circuit coupled with the control circuit and configured to provide two-way data communication external to the IMD.

4. The IMD of claim 2, wherein the telemetry circuit is an RF transceiver.

5. The IMD of claim 3, wherein the telemetry circuit has a range of up to about 30 meters.

6. The IMD of claim 3, wherein the actuator is a subcutaneous switch disposed on an exterior of the housing.

7. The IMD of claim 6, wherein actuation of the subcutaneous switch a first predetermined number of times causes the control circuit to store data in the memory.

8. The IMD of claim 7, wherein actuation of the subcutaneous switch a second predetermined number of time causes the control circuit to telemeter data from the memory.

9. The IMD of claim 8, wherein actuation of the subcutaneous switch a third predetermined number of time causes the control circuit to telemeter a message requesting emergency assistance.

10. The IMD of claim 3, wherein actuator is an motion sensor.

11. The IMD of claim 3, wherein the actuator is an accelerometer.

12. The IMD of claim 11, wherein tapping of the accelerometer cases actuation.

13. The IMD of claim 12, wherein actuation of the accelerometer a first predetermined number of times causes the control circuit to store data in the memory.

14. The IMD of claim 13, wherein actuation of accelerometer a second predetermined number of time causes the control circuit to telemeter data from the memory.

15. The IMD of claim 8, wherein actuation of the accelerometer a third predetermined number of time causes the control circuit to telemeter a message requesting emergency assistance.

16. An subcutaneously implantable loop recorder for recording cardiac data comprising:
a housing;
a control circuit disposed within the housing;
at least two electrodes disposed on an outer surface of the housing for sensing the cardiac data;
a memory coupled with the control circuit and configured to selective store the cardiac data;
a telemetry circuit operatively coupled with the control circuit and configured to telemeter data external to the recorder;
a patient actuable mechanical input mechanism disposed within the housing and configured to provide data input to the control circuit post-implant.

17. The loop recorder of claim 16, wherein actuation of the input mechanism a first predetermined number of times causes the control circuit to record a predetermined amount of cardiac data into the memory.

18. The loop recorder of claim 17, wherein actuation of the input mechanism a second predetermined number of times causes the control circuit to telemeter data from the memory.

19. The loop recorder of claim 18, wherein actuation of the input mechanism a third predetermined number of times causes the control circuit to telemeter a message summoning an emergency response.

20. The loop recorder of claim 16, wherein the input mechanism is an accelerometer.
